SEQUENCE LISTING (2 sequences)

SEQ ID NO: 1 - DNA, 1749 bp, Homo sapiens
gagggcgcccccaccccacccgcccaccctccccgcttctcgctaggtcccgattggctggcgcgctccgcggctgggatggcagtgggaggggaccctctttcctaacggggttataaaaacagcgccctcggcggggtccagtcctctgccactctcgctccgaggtccccgcgccagagacgcagccgcgctcccaccacccacacccaccgcgccctcgttcgcctcttctccgggagccagtccgcgccaccgccgccgcccagcccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcgacgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctaggggacctctacgaggaggagatgcgggagctgcgccggcaggtggaccagctaaccaacgacaaagcccgcgtcgaggtggagcgcgacaacctggccgaggacatcatgcgcctccgggaaaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacattcctgattaagacggttgaaactagagatggacaggttatcaacgaaacttctcagcatcacgatgaccttgaataaacaattgcacactcagtgcagcactcatataccagcagataaaagaatccatatctt

SEQ ID NO: 2 - protein, 466 aa, Homo sapiens [sequence omitted]

(19) United States
(12) Patent Application Publication
(10) Pub. No.: US 2010/0094026 A1
(43) Pub. Date: Apr. 15, 2010

(54) METHOD OF SCREENING COMPOUND USEFUL IN TREATING ALLERGIC DISEASE

(75) Inventors: Akito Tanaka, Tsukuba-shi (JP); Akinobu Nakanishi, Hino-shi (JP); Masayuki Haramura, Kamakura-shi (JP); Mikio Takeuchi, Tsukuba-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: REVERSE PROTEOMICS RESEARCH INSTITUTE CO., LTD., Tokyo (JP)

(21) Appl. No.: 12/092,208

(22) PCT Filed: Aug. 11, 2006
(86) PCT No.: PCT/JP2006/316200

§ 371 Date: Dec. 7, 2009

(30) Foreign Application Priority Data
Nov. 1, 2005 (JP) 2005-318833

(51) Int. Cl. A61K 31/35; C07D 407/12; G01N 33/566

(52) U.S. Cl. 549/402; 436/501

(57) ABSTRACT

The present invention provides a method of screening for a compound useful for treatment of an allergic disease by identifying a molecule targeted by Intal upon exertion of its efficacy as an anti-allergic agent (hereinafter referred as a target) and using the target, as well as a new type of anti-allergic agent comprising a compound obtainable by the screening as an active ingredient for treatment of an allergic disease.

### TECHNICAL FIELD

The present invention relates to a method of screening for a compound useful for the treatment of an allergic disease and a pharmaceutical composition used for treatment of an allergic disease. More specifically, the present invention relates to a method of screening for a compound useful for the treatment of an allergic disease by identifying a molecule targeted by Intal upon exertion of its efficacy as an anti-allergic agent (hereinafter referred as a target) and using the target.

### BACKGROUND OF THE INVENTION

As the base sequences of the human genome is progressively determined, the object of the research is shifting to genomic drug discovery, a search and/or identification of a target in the drug discovery. Among them, the identification of a target of Intal, an anti-allergic agent, also is drawing the attention as one of the object of the research.

It is known that Intal has an action to suppress the release (i.e. degranulation) of chemical mediators such as histamine and SRS-A from mast cells which occurs upon antigen-antibody reaction (e.g., see Chiryoyaku manual 2004, Igaku-Shoin, p 301), and the like.

To date, regarding its mechanism of action, a mechanism inhibiting the secretion of histamine and the like due to phospholipase A stimulation (e.g., see Orr T S. et al., Nature, 1969, 223 (202), pp. 197-198); a mechanism in which a protein playing a role in a calcium channel is involved (e.g., see Mazurek N. et al., Proceedings of the National Academy of Sciences USA., 1984, 81 (21), pp. 6841-6845); a mechanism in which a kinase for intracellular proteins is involved (e.g., Theoharides T C. et al., Science, 1980, 207 (4426), pp. 80-82) and the like has been reported.

However, in spite of a great deal of efforts as described above, the identification of a target that Intal directly acts on and whose pharmacological activity can be fully explained, has been an unresolved problem.

Therefore, it has been very difficult to create a pharmaceutical agent which has an effect over Intal or is alternative thereto. Thus, to create the pharmaceutical agent, it is needed to develop an effective method of screening for a compound having a mechanism of action and pharmacologic activity similar to Intal.

### DISCLOSURE OF THE INVENTION

The object of the present invention is to provide a method of screening for a compound useful for the treatment of an allergic disease by identifying a molecule targeted by Intal upon exertion of its efficacy as an anti-allergic agent and using the target, as well as a new type of pharmaceutical composition for treatment of an allergic disease comprising a compound obtainable by the screening as an active ingredient.

The present inventors searched for a protein to which Intal specifically binds to solve the above described problems and as a result, found out that Vimentin, one of intermediate filaments, specifically binds to Intal. It is known that there are Ser residues that are regiospecifically phosphorylated by cdc 2 kinase, PKA, PKC, CaMK II, Rho kinase and the like in the head part of Vimentin (e.g., Signal Transduction, pp. 235-241). From the fact, the present inventors assumed that phosphorylation of Vimentin would be involved in the intracellular signal transduction in the degranulation, and as a result of further diligent investigation, developed a method of screening a compound useful for the treatment of an allergic disease by using Vimentin, which resulted in the completion of the present invention.

That is, the present invention is as following:

[1] A method of screening for a compound useful for the treatment of an allergic disease, which comprises a step of determining whether a test compound specifically binds to Vimentin or a functional fragment thereof.
[2] A method of screening for a compound useful for the treatment of an allergic disease, which comprises the following steps:
(1) contacting Vimentin or a functional fragment thereof with test compounds,
(2) determining whether the test compounds specifically bind to Vimentin or a functional fragment thereof, and
(3) selecting a test compound which specifically binds to Vimentin or a functional fragment thereof in the step (2) above.
[3] A method of screening for a compound useful for the treatment of an allergic disease, which comprises the following steps:
(1) contacting a protein having an amino acid sequence of SEQ ID NO:2 or a fragment thereof with test compounds,
(2) determining whether the test compounds specifically bind to the protein or a functional fragment thereof, and
(3) selecting a test compound which specifically binds to the protein or a functional fragment thereof in the step (2) above.
[4] A method of screening for a compound useful for the treatment of an allergic disease, which comprises the following steps:
(1) contacting, with test compounds, a protein having an amino acid sequence with deletion, substitution or addition of one or more amino acids in the amino acid sequence of SEQ ID NO:2 and binding to the following compound

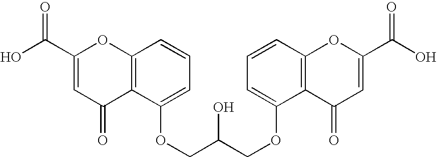

or a fragment thereof,
(2) determining whether the test compounds specifically bind to the protein or a functional fragment thereof, and
(3) selecting a test compound which specifically binds to the protein or a functional fragment thereof in the step (2) above.
[5] A compound useful for the treatment of an allergic disease, obtainable by the method of screening according to any one of the aforementioned [1] to [4].
[6] A pharmaceutical composition comprising a compound which specifically binds to Vimentin as an active ingredient.
[7] A pharmaceutical composition comprising a compound which controls the expression of Vimentin as an active ingredient.
[8] A pharmaceutical composition comprising a compound which controls the activity of Vimentin as an active ingredient.
[9] The pharmaceutical composition according to any one of the aforementioned [6] to [8], which is used for the treatment of an allergic disease.
[10] A compound represented by the formula (I):

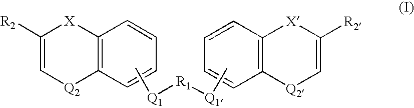

[wherein,
R₁ is optionally substituted divalent hydrocarbon;
R₂ and R_{2′} are the same or different and each is carboxylic acid, amide or ester (which is optionally substituted by lower alkyl) or tetrazole;
Q₁ and Q_{1′} are the same or different and each is O, S, NH or CH₂;
Q₂ and Q_{2′} are the same or different and each is C═O, C═S, C═NH, C═NOH, C═NOR₃, O, alkylene or NH;
X is CH₂, O, S, NH or NR₃ (wherein, R₃ is optionally substituted alkyl or cycloalkyl)],
or a pharmaceutically acceptable salt thereof, provided that the following compound is excluded:

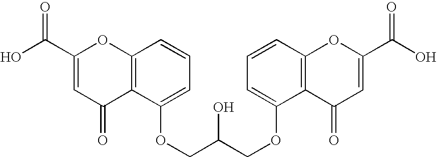

[11] A therapeutic agent for an allergic disease, which comprises the compound according to the aforementioned [10] or a pharmaceutically acceptable salt thereof.

The present invention provides a method of screening for a compound useful for the treatment of an allergic disease by using Vimentin, as well as a new type of anti-allergic agent comprising a compound obtainable by the screening as an active ingredient.

### BRIEF DESCRIPTION OF THE DRAWINGS

FIG. 1 shows results of binding experiment in Example 2, demonstrating the specific binding between Intal and Vimentin.

### BEST MODE FOR CARRYING OUT THE INVENTION

Hereinafter, the contents of the present invention will be explained in detail.

Herein, by “nucleic acid molecule” is meant a single strand or double strand DNA or RNA. Herein, unless otherwise specified, by “nucleotide sequence” is meant a sequence of deoxyribonucleotide (expressed by A, G, C and T) or a sequence of ribonucleotide (expressed by A, G, C and U). Herein, unless otherwise specified, a single strand nucleotide sequence is shown with 5′ terminal at the left end and 3′ terminal at the right end.

Herein, unless otherwise specified, for the representation of amino acid, 1-letter or 3-letter abbreviation which is a standard representation regarding amino acids is used. Herein, unless otherwise specified, an amino acid sequence is shown with N terminal (amino terminal) at the left end and C terminal (carboxyl terminal) at the right end.

Herein, by “Vimentin” used as a target protein of the screening is, preferably, meant Vimentin derived from human. Human Vimentin includes, for example, a protein having an amino acid sequence represented by GenPept Accession No. AAA61279 (SEQ ID NO: 2), but as long as it can specifically bind to Intal (that is, it can play a role as a target of Intal), may be a protein having an amino acid sequence with deletion, substitution and/or addition of one or two or more (preferably, about 1-30, preferably about 1-10, more preferably 1-5) amino acids in the amino acid sequence of SEQ ID NO: 2.

Alternatively, human Vimentin includes, for example, a protein which is represented by an amino acid sequence having a homology of 60% or more, 70% or more, 80% or more, preferably 90% or more, and especially preferably 95% or more with the amino acid sequence of SEQ ID NO:2, and can specifically bind to Intal.

As used herein, “homology” means the degree of sequence correlation between two polypeptide sequences. A large number of methods of measuring the homology between two polypeptide sequences are known, and the term “homology” (also called “identity”) is obvious to those skilled in the art. For example, ordinary methods used to measure the homology of two sequences include, but are not limited to, those disclosed in Martin, J. Bishop (Ed.), Guide to Huge Computers, Academic Press, San Diego (1994); Carillo, H. & Lipman, D., SIAM J. Applied Math., 48:1073 (1988) and the like.

As a preferable method for measuring the homology, one designed to obtain the largest matching portion between the two sequences tested can be mentioned. As such a method, one assembled in a computer program can be mentioned. Preferable computer programming methods for measuring the homology between two sequences include, but are not limited to, the GCG program package (Devereux, J. et al., Nucleic Acids Research, 12(1):387 (1984)), BLASTP, FASTA and the like; methods known in the art can be used.

In addition, “Vimentin” used as a target protein of the screening in the present invention may be any fragment of the Vimentin as long as it has a property to specifically bind to Intal (hereinafter, such a fragment is also referred to as “functional fragment”).

Intal may be used to confirm whether Vimentin or a functional fragment thereof specifically bind to Intal or not. Intal is commercially available or can also be produced according to a known technology.

Herein, “specifically bind” is exemplified by the relation of a specific receptor to an agonist or an antagonist, the relation of an enzyme to a substrate, and the relation of, for example, an FK506-binding protein (target molecule) to FK506 (ligand), a steroid hormone receptor to a steroid hormone (e.g., dexamathason and glucocorticoid receptor), HDAC to the anticancer agent trapoxin, and the like, and can be confirmed as numerical values of Kd, Ka and the like by competitive experiments and the like. The specific bind can also be confirmed by a visual means such as electrophoresis, in addition to representation as the aforementioned specific numerical values.

The present invention also provides a pharmaceutical composition comprising a compound that specifically binds to Vimentin and a functional fragment thereof as an active ingredient.

A compound capable of binding to Vimentin or a functional fragment thereof, like Intal, can exhibit excellent anti-allergic action on human or non-human mammals (e.g., monkey, horse, bovine, sheep, dog, cat, rabbit, mouse, rat, guinea pig and the like). Therefore, the pharmaceutical composition of the present invention is useful as a therapeutic agent for various allergic diseases (e.g., bronchial asthma, allergic rhinitis, atopic dermatitis, pollinosis and the like).

Moreover, a compound which controls directly or indirectly the expression or activity of Vimentin can also be useful for the treatment of allergic diseases.

A compound or substance which controls the expression or activity of Vimentin includes DNA encoding Vimentin, a vector in which DNA encoding Vimentin is inserted, a Vimentin protein and the like.

A compound capable of binding to Vimentin or a functional fragment thereof includes, for example, a compound represented by the following formula (I) or a pharmaceutically acceptable salt thereof (hereinafter, collectively also referred as “compound (I)”).

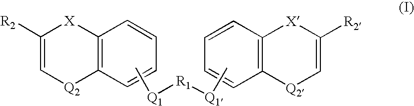

[wherein,
R₁ is substituted divalent hydrocarbon;
R₂ and R_{2′} are the same or different and each is carboxylic acid, amide or ester (which is optionally substituted by lower alkyl) or tetrazole;
Q₁ and Q_{1′} are the same or different and each is O, S, NH or CH₂;
Q₂ and Q_{2′} are the same or different and each is C═O, C═S, C═NH, C═NOH, C═NOR₃, O, CH₂ or NH;
X is CH₂, O, S, NH or NR₃ (wherein, R₃ is optionally substituted alkyl or cycloalkyl)].

The above-mentioned “divalent hydrocarbon group” includes, for example, a “divalent acyclic hydrocarbon group”, “divalent cyclic hydrocarbon group”, or divalent group obtainable with combination of one or more “divalent acyclic hydrocarbon group” and one or more “divalent cyclic hydrocarbon group”.

As used herein, “divalent acyclic hydrocarbon group” includes, for example, alkylene having a carbon number of 1 to 20, alkenylene having a carbon number of 2 to 20, alkynylene having a carbon number of 2 to 20 and the like.

“Divalent cyclic hydrocarbon group” includes, a divalent group obtainable by removing any two hydrogen atoms from cycloalkane having a carbon number of 5 to 20, cycloalkene having a carbon number of 5 to 20 or aromatic hydrocarbon having a carbon number of 6 to 18 (e.g., benzene, naphthalene, inden, anthracene and the like) and the like. Specific example thereof includes 1,2-cyclopentylene, 1,3-cyclopentylene, 1,2-cyclohexylene, 1,3-cyclohexylene, 1,4-cyclohexylene, 1,2-cycloheptylene, 1,3-cycloheptylene, 1,4-cycloheptylene, 3-cyclohexen-1,4-ylene, 3-cyclohexen-1,2-ylene, 2,5-cyclohexadien-1,4-ylene, 1,2-phenylene, 1,3-phenylene, 1,4-phenylene, 1,4-naphthylene, 1,6-naphthylene, 2,6-naphthylene, 2,7-naphthylene, 1,5-indenylene, 2,5-indenylene and the like.

The above-mentioned “lower alkyl” denotes, for example, linear or branched alkyl group having a carbon number of 1 to 6, and includes alkyl group having a carbon number of 1 to 10 such as methyl, ethyl, n-propyl, isopropyl, butyl, isobutyl, sec-butyl, tert-butyl, n-pentyl, isopentyl, neopentyl, n-hexyl, isohexyl, and the like.

The above-mentioned “cycloalkyl group” denotes, for example, cyclic alkyl group having a carbon number of 3 to 6, and includes cyclopropyl, cyclobutyl, cyclopentyl, cyclohexyl and the like.

Substituent of the above-mentioned “optionally substituted alkyl or cycloalkyl” includes, but not particularly limited to, for example, saturated or unsaturated cyclic hydrocarbon group, saturated or unsaturated heterocyclic group, halogen atom, cyano group, nitro group, hydroxyl group, optionally substituted carboxyl group, optionally substituted amide group, optionally substituted lower alkyl group, optionally substituted aroxy group, optionally substituted amino group, optionally substituted alkoxy group and the like.

These substituents are substituted on the hydrocarbon chain group within a chemically acceptable range. Provided that, when the number of the substituents is two or more, the substituents may be the same or different from each other.

“Saturated or unsaturated cyclic hydrocarbon group” includes saturated or unsaturated cyclic hydrocarbon group having a carbon number of 3 to 18, specifically, for example, alicyclic hydrocarbon group, aromatic hydrocarbon group and the like.

“Alicyclic hydrocarbon group” includes, for example, monocyclic or fused polycyclic group composed of 3 to 10 carbon atoms, specifically cycloalkyl group, cycloalkenyl group, and bicyclic or tricyclic fused ring of the group and aryl group having a carbon number of 6 to 14 (e.g., benzene etc.) and the like, etc. The “cycloalkyl group” includes, for example, cycloalkyl group having a carbon number of 3 to 6 such as cyclopropyl, cyclobutyl, cyclopentyl, cyclohexyl, and the like. The “cycloalkenyl group” includes, for example, cycloalkenyl group having a carbon number of 3 to 6 such as cyclopropenyl, cyclobutenyl, cyclopentenyl, cyclohexenyl, and the like.

“Aromatic hydrocarbon group” includes, for example, monocyclic aromatic hydrocarbon group, fused polycyclic aromatic hydrocarbon group composed of 6 to 18 carbon atoms and the like, and specifically, aryl group having a carbon number of 6 to 14 such as phenyl, 1-naphthyl, 2-naphthyl, 2-indenyl, 2-anthryl and the like.

The cyclic hydrocarbon group may be optionally substituted by substituent(s) such as saturated or unsaturated cyclic hydrocarbon group, saturated or unsaturated heterocyclic group, halogen atom, cyano group, nitro group, oxo group, optionally substituted carboxyl group, substituted amide group, optionally substituted lower alkyl group, optionally substituted amino group, optionally substituted alkoxy group, and these substituents are substituted on the cyclic hydrocarbon group within a chemically acceptable range. Provided that, when the number of the substituents is two or more, the substituents may be the same or different from each other.

“Saturated or unsaturated heterocyclic group” includes, for example, 5 to 6-membered monocyclic group comprising 1 to 2 nitrogen atoms, 5 to 6-membered monocyclic group comprising 1 to 2 nitrogen atoms and 1 oxygen atom or 1 sulfur atom, 5-membered monocyclic group comprising 1 oxygen atom or 1 sulfur atom, bicyclic group comprising 1 to 4 nitrogen atoms and formed by condensation of 6-membered ring and 5 or 6-membered ring, and the like, and specifically includes, for example, pyridyl, thienyl, oxadiazolyl, imidazolyl, thiazolyl, isothiazolyl, oxazolyl, isoxazolyl, furyl, pyrrolyl, quinolyl, quinazolinyl, purinyl, pyrazolyl, thiophenyl and the like. The heterocyclic group may be optionally substituted by substituent(s) such as saturated or unsaturated cyclic hydrocarbon group, saturated or unsaturated heterocyclic group, halogen atom, cyano group, nitro group, oxo group, optionally substituted carboxyl group, substituted amide group, optionally substituted lower alkyl group, optionally substituted amino group, optionally substituted alkoxy group, and these substituents are substituted on the heterocyclic group within a chemically acceptable range. When the number of the substituents is two or more, the substituents may be the same or different from each other.

“Halogen atom” includes fluorine, chlorine, bromine and iodine.

“Optionally substituted carboxyl group” includes carboxyl group optionally substituted by lower alkyl group, lower alkanoyl group (e.g., alkanoyl group having a carbon number of 1 to 6 such as formyl, acetyl, propionyl) and the like, etc.

“Optionally substituted amide group” includes, unsubstituted amide group, substituted amide [N-substituted amide group or N,N′-disubstituted amide group, specifically amide group substituted by lower alkyl group and the like].

“Lower alkyl group” denotes, for example, linear, branched or cyclic alkyl group having a carbon number of 1 to 6, and specifically includes methyl, ethyl, n-propyl, isopropyl, butyl, isobutyl, sec-butyl, tert-butyl, pentyl, hexyl, cyclopropyl, cyclobutyl and the like.

“Substituent” of the “optionally substituted lower alkyl group” includes, carboxyl group, substituted amide group, cyano group, hydroxyl group, halogen atom and the like.

“Aroxy group” includes, for example, 6-membered monocyclic group (e.g., phenyl group) with an interposed oxygen atom, and specifically includes, for example, phenoxy and the like.

The monocyclic group is optionally substituted by substituent(s) such as saturated or unsaturated cyclic hydrocarbon group, saturated or unsaturated heterocyclic group, halogen atom, cyano group, nitro group, optionally substituted carboxyl group, substituted amide group, optionally substituted lower alkyl group, optionally substituted amino group, optionally substituted alkoxy group, and these substituents are substituted on the ring group within a chemically acceptable range. When the number of the substituents is two or more, the substituents may be the same or different from each other.

Further, the monocyclic group may form a fused ring by condensation with saturated or unsaturated cyclic hydrocarbon group or saturated or unsaturated heterocyclic group. The fused ring includes inden, naphthalene, fluorein, phenanthrene, anthracene, indole, isoindole, benzofuran, benzothiophene, indolizine, chromene, quinoline, isoquinoline, indazole, quinazoline, cinnoline, quinoxaline, phthalazine and the like.

“Optionally substituted amino group” includes, amino group optionally substituted by lower alkyl group, lower alkanoyl group (e.g., alkanoyl group having a carbon number of 1 to 6 such as formyl, acetyl, propionyl) and the like, etc.

By “Alkoxy group” is meant straight or branched alkoxy group having a carbon number of 1 to 6, and specifically includes methoxy group, ethoxy group, n-propoxy group, isopropoxy group, n-butoxy group, isobutoxy group, sec-butoxy group, tert-butoxy group, n-pentyloxy group, isopentyloxy group, tert-pentyloxy group, neopentyloxy group, 2-pentyloxy group, 3-pentyloxy group, n-hexyloxy group, 2-hexyloxy group and the like.

Substituent of the “optionally substituted alkoxy group” includes, but is not particularly limited to, for example, saturated or unsaturated cyclic hydrocarbon group, saturated or unsaturated heterocyclic group, halogen atom, cyano group, nitro group, hydroxyl group, optionally substituted carboxyl group, substituted amide group, optionally substituted lower alkyl group, optionally substituted aroxy group, optionally substituted amino group, optionally substituted alkoxy group and the like. These substituents are substituted on the alkoxy group within a chemically acceptable range. Provided that, when the number of the substituents is two or more, the substituents may be the same or different from each other.

A compound represented by the formula (I) of the present invention can be produced by taking advantage of a property based on a type of the basic backbone or substituent thereof and applying various known synthesis methods. For example, such a method includes alkylation, acylation, amination, imination, halogenation, reduction, oxidation, condensation and the like, and reactions and methods commonly used in the art can be utilized.

The compound represented by the formula (I) of the present invention may have formed a pharmaceutically acceptable salt; as the salt, acid addition salts, for example, inorganic acid salts (e.g., hydrochlorides, sulfates, hydrobromates, phosphates and the like), organic acid salts (e.g., acetates, trifluoroacetates, succinates, maleates, fumarates, propionates, citrates, tartrates, lactates, oxalates, methanesulfonates, p-toluenesulfonates and the like) and the like can be mentioned.

Note that the compound represented by the formula (I) of the present invention or a salt thereof may be a solvate such as a hydrate.

When the compound represented by the formula (I) of the present invention and a compound obtainable by a method of screening of the present invention (collectively, also referred as “compound of the present invention”) are used as a therapeutic drug for an allergic disease, it can be prepared as an ordinary pharmaceutical preparation and administered orally or parenterally.

For oral administration, the compound of the present invention can be administered in a dosage form in common use in the art. For parenteral administration, the compound of the present invention can be administered in a dosage form such as a topical preparation (transdermal preparation and the like), a rectal preparation, an injection, or a nasal preparation.

As examples of the oral preparation or rectal preparation, capsules, tablets, pills, powders, drops, cachets, suppositories, liquids and the like can be mentioned.

As examples of the injection, a sterile solution or suspension and the like can be mentioned. As examples of the topical preparation, creams, ointments, lotions, transdermal preparations (ordinary patches and matrices) and the like can be mentioned.

The above-described dosage forms can be formulated along with a pharmaceutically acceptable excipient and additive by a technique commonly performed in the art. As the pharmaceutically acceptable excipient and additive, carriers, binders, flavoring agents, buffering agents, thickeners, colorants, stabilizers, emulsifiers, dispersing agents, suspending agents, antiseptics and the like can be mentioned.

As examples of pharmaceutically acceptable carriers, magnesium carbonate, magnesium stearate, talc, sugar, lactose, pectin, dextrin, starch, gelatin, gum tragacanth, methylcellulose, sodium carboxymethylcellulose, low-melting-point waxes, cacao butter and the like can be mentioned.

The tablets can be prepared as tablets with ordinary coatings, for example, sugar-coated tablets, enteric coated tablets, film-coated tablets, and double-layered tablets or multilayered tablets if necessary.

The powders are formulated into preparations along with a pharmaceutically acceptable base for powders. As the base, talc, lactose, starch and the like can be mentioned.

The drops can be formulated into preparations along with an aqueous or non-aqueous base and one or more kinds of pharmaceutically acceptable diffusing agents, suspending agents, solubilizers and the like.

The capsules can be produced by filling therein an active ingredient compound, along with a pharmaceutically acceptable carrier. The compound can be mixed with a pharmaceutically acceptable excipient and filled in the capsules, or filled without an excipient.

The caches can also be produced in the same manner. When the present invention is prepared as a suppository, it is formulated into preparations by a commonly used technique along with a base such as a vegetable oil (castor oil, olive oil, peanut oil and the like), a mineral oil (petrolatum, white petrolatum and the like), a wax, or a partially synthesized or totally synthesized glycerine fatty acid ester.

As the liquid for injection, solutions, suspensions, emulsions and the like can be mentioned. For example, aqueous solutions, water-propylene glycol solutions and the like can be mentioned. The liquid can also be produced in the form of a solution of polyethylene glycol and/or propylene glycol that may contain water.

A liquid suitable for oral administration can be produced by adding an active ingredient compound to water and, if required, adding a colorant, flavoring agent, stabilizer, sweetener, solubilizer, thickener and the like. A liquid suitable for oral administration can also be produced by adding the compound, along with a dispersing agent, to water to increase the viscosity. As examples of the thickener, pharmaceutically acceptable natural or synthetic rubbers, resins, methylcellulose, sodium carboxymethylcellulose, known suspending agents and the like can be mentioned.

As the topical preparation, the above-described liquids, as well as creams, aerosols, sprays, dusting powders, lotions, ointments and the like can be mentioned. The above-described topical preparation can be produced by mixing an active ingredient compound and pharmaceutically acceptable diluent and pharmaceutically acceptable carrier. Ointments and creams are prepared by, for example, adding a thickener and/or a gelling agent to an aqueous or oily base. As examples of the base, water, liquid paraffin, vegetable oils and the like can be mentioned. As examples of the thickener, soft paraffin, aluminum stearate, cetostearyl alcohol, propylene glycol, polyethylene glycol, lanolin, hydrogenated lanolin, beeswax and the like can be mentioned. To the topical preparation, an antiseptic such as methyl hydroxybenzoate, propyl hydroxybenzoate, chlorocresol, or benzalkonium chloride, and a bacterial growth inhibitor can be added. A lotion can be prepared by adding one or more kinds of pharmaceutically acceptable stabilizers, suspending agents, emulsifiers, diffusing agents, thickeners, colorants, flavoring agents and the like to an aqueous or oily base.

Dosage and frequency of administration vary depending on the kind of compound to be used, symptoms, age and body weight of patients, dosage form and the like, and are set as appropriate according to them.

The present invention also provides a screening method for a compound useful for the treatment of an allergic disease, with specific bindability to Vimentin or a functional fragment thereof as an index.

“Test compound” used in the present invention may be any known compound or novel compound, and includes, but not particularly limited to, for example, nucleic acid, carbohydrates, lipid, protein, peptide, antibody, organic or inorganic low-molecular-weight compound, organic or inorganic polymer compound, compound library produced by combinatorial chemistry techniques, random peptide library produced by solid phase synthesis or phage display method, or natural component derived from microorganisms, animals or plants, and the like.

In the present screening method, Vimentin or a functional fragment thereof can be used as a purified or unpurified protein (polypeptide) or a (functional) fragment thereof, and can be used in the state of being expressed in cells.

Vimentin or a (functional) fragment thereof can be acquired by using as appropriate a known technique such as (1) a method comprising isolation and purification from a cell culture or tissue, as a starting material, producing Vimentin or a (functional) fragment thereof, (2) a method comprising chemical synthesis, or (3) a method comprising purification from cells manipulated by gene recombination technology and the like to express Vimentin or a (functional) fragment thereof.

Isolation and purification of the Vimentin or a (functional) fragment thereof of the present invention can, for example, be performed as described below. That is, the Vimentin or a (functional) fragment thereof is extracted and purified by a known method from a tissue expressing Vimentin or a (functional) fragment thereof, or a culture obtained by culturing cells expressing Vimentin or a (functional) fragment thereof in an appropriate liquid medium. For the extraction and purification, known methods are used as appropriate depending on the fraction wherein the desired product is present.

Specifically, the method is performed as described below.

First, a tissue or culture is directly subjected to a conventional method such as filtration or centrifugation, and the tissue or cells or the supernatant is recovered. If the desired protein has been accumulated in the cells, the recovered cells are suspended in an appropriate buffer solution, and a surfactant is added at an appropriate concentration to solubilize the membrane. As the surfactant, sodium dodecyl sulfate (SDS), cetyltrimethylammonium bromide (CTAB) and the like can be mentioned. Since these exhibit potent protein denaturing action, it is preferable to use a gently acting nonionic surfactant, for example, Triton X-100 and the like, to ensure that the protein is folded to show biological activity.

Next, the crude extract obtained is treated in the presence of a surfactant if required, using commonly used methods in combination as appropriate, to isolate and purify the protein or a functional fragment thereof. As the commonly used methods, for example, methods utilizing solubility, such as salting-out and solvent precipitation; methods utilizing difference in molecular weight, such as dialysis, ultrafiltration, gel filtration, and SDS-PAGE; methods utilizing electric charge, such as ion exchange chromatography; methods utilizing specific affinity, such as affinity chromatography; methods utilizing difference in hydrophobicity, such as reverse phase high performance liquid chromatography; methods utilizing difference in isoelectric point, such as isoelectric focusing; and the like can be mentioned. More specifically, the protein or a functional fragment thereof can be separated and purified by commonly used methods, for example, concentration under reduced pressure, lyophilization, extraction with conventionally used solvents, pH adjustment, treatment with conventionally used adsorbents such as anion exchange resin or cation exchange resin, and nonionic adsorption resin, crystallization, recrystallization and the like.

Production of the Vimentin or a (functional) fragment thereof of the present invention by chemical synthesis can be performed by, for example, synthesis or semi-synthesis based on the amino acid sequence information shown by SEQ ID NO:2 using a peptide synthesizer.

Also, when the Vimentin or a (functional) fragment thereof is acquired from cells manipulated to express the same by gene recombination technology and the like, the specific procedures are performed as described below.

First, an expression vector that functionally carries the gene encoding Vimentin or a functional fragment thereof is prepared.

The gene that encodes Vimentin or a functional fragment thereof may be obtained by any method. For example, a complementary DNA (cDNA) prepared from an mRNA, a genomic DNA prepared from a genomic library, a chemically synthesized DNA, a DNA obtained by amplification by the PCR method with an RNA or DNA as a template, and a DNA constructed by appropriately combining these methods, and the like are included.

For example, a DNA comprising all or a portion of a DNA substantially comprising a base sequence of the human Vimentin entire coding region shown by SEQ ID NO:1 (GenBank Accession No. M14144), and the like can be mentioned. Also, a technology for substituting or deleting an optionally chosen base in the above-described base sequence (e.g., in vitro mutagenesis, site-directed mutagenesis and the like) can also be utilized.

As used herein, “a DNA substantially comprising” means, in addition to the above-described DNAs comprising a particular base sequence, a DNA comprising a base sequence capable of hybridizing to the above-described DNAs comprising a particular base sequence under stringent conditions (in the present invention, these conditions refer to conditions under which a DNA having a homology of about 60% or more, preferably about 80% or more, and more preferably about 90% or more, in terms of base sequence can hybridize; stringency can be controlled by changing the temperature, salt concentration and the like as appropriate during the hybridization reaction and washing).

Stringent conditions can be calculated on the basis of the desired homology, the length of oligonucleotide and the like by applying them to appropriate calculation formulas utilized in the art. For example, hybridization at 42° C. and washing treatment at 42° C. with a buffer solution containing 1×SSC and 0.1% SDS, hybridization at 65° C. and washing treatment at 65° C. with a buffer solution containing 0.1×SSC and 0.1% SDS, and the like can be mentioned.

An expression vector that functionally comprises a gene encoding Vimentin or a functional fragment thereof can be obtained by inserting the DNA obtained into a plasmid vector, phage vector or the like capable of retaining replication or autonomous replication in various hosts of prokaryotic cells and/or eukaryotic cells by means of an appropriate restriction endonuclease site.

As used herein, “functionally” means that the gene (DNA) is arranged to allow transcription in a host cell matching with the vector, and to allow the production of the protein encoded thereby. Preferably, the expression vector is a vector having an expression cassette wherein a promoter region, an initiation codon, a gene encoding Vimentin or a functional fragment thereof, a stop codon and a terminator region are continuously arranged. For transformant selection, it is preferable that a selection marker gene be further contained.

For example, when a mammalian cell is transformed, a plasmid comprising a promoter and a polyadenylation signal both of an animal virus, for example, SV40, RSV, MMLV and the like, joined to each other via a restriction endonuclease site, preferably a multicloning site, wherein a selection marker gene derived from a plasmid such as pSV2-neo or pSV2-dhfr (neomycin resistance gene, dihydrofolate reductase and the like) has been inserted, can be used.

The host cell is not subject to limitation, as long as it matches with the expression vector used, and is transformable; various cells in common use in the technical field of the present invention, such as natural cells or an artificially established line of recombinant cells and the like, can be utilized. Specifically, bacteria such as Escherichia coli and Bacillus subtilis, fungi such as yeast, animal cells or insect cells and the like can be mentioned as examples. Preferably, mammalian cells, particularly rat-derived cells, hamster-derived cells (CHO, BHK and the like), mouse-derived cells (COP, L, C127, Sp2/0, NS-1, NIH T3 and the like), monkey-derived cells (COS1, COS3, COS7, CV1, Velo and the like) and human-derived cells (HeLa, diploid fibroblast-derived cells, myeloma cells, Namalwa, Jurkat cells and the like) can be mentioned.

Introduction of an expression vector to a host cell can be performed using a conventionally known method. For example, when the expression vector is introduced to a mammalian cell, the calcium phosphate co-precipitation method, the protoplast fusion method, the microinjection method, the electroporation method, the lysosome method and the like can be mentioned.

Vimentin or a functional fragment thereof can also be produced by culturing a transformant comprising an expression vector prepared as described above. The medium preferably contains a carbon source and inorganic or organic nitrogen source required for the growth of the host cell (transformant). As examples of the carbon source, glucose, dextrin, soluble starch, sucrose and the like can be mentioned; as examples of the nitrogen source, ammonium salts, nitrates, amino acids, corn steep liquor, peptone, casein, meat extract, soybean cake, potato extract and the like can be mentioned. If desired, other nutrients [for example, inorganic salts (calcium chloride, sodium dihydrogen phosphate, magnesium chloride and the like), vitamins, antibiotics (tetracycline, neomycin, kanamycin, ampicillin and the like)] may be contained.

The cultivation is performed by a method known in the art. The cultivation conditions are conditions enabling the expression of the protein; for example, temperature, medium pH and cultivation time are chosen as appropriate so that the protein is produced in a large amount.

For example, when the host is an animal cell, as examples of the medium, a minimum essential medium (MEM) containing about 5 to 20% fetal calf serum (FCS), Dulbecco's modified Eagle medium (DMEM), RPMI-1640 medium, 199 medium and the like can be used. The pH of the medium is preferably about 6 to 8, the cultivation is normally performed at 30 to 40° C. for about 15 to 72 hours, and the culture may be aerated or agitated as necessary.

The Vimentin or a functional fragment thereof of the present invention can be collected from the culture obtained from the above-described cultivation in the same manner as the aforementioned extraction, isolation, and purification from cells or tissues expressing Vimentin or a functional fragment thereof.

Contact treatment of the Vimentin or a functional fragment thereof thus obtained and a test compound can be performed in accordance with a binding experiment commonly performed in the art. Specifically, in cases where Vimentin or a functional fragment thereof or a test compound is immobilized to a solid phase carrier, a solution comprising the test compound is brought into contact with the solid phase carrier when the Vimentin or a functional fragment thereof is immobilized, and a solution comprising the Vimentin or a functional fragment thereof (a purified protein solution or a crudely purified protein solution such as cell extract or tissue extract) is brought into contact with the solid phase carrier when the test compound is immobilized to the solid phase carrier. The column method, the batch method and the like can be utilized.

The step for determining whether or not the test compound binds specifically to Vimentin or a functional fragment thereof can be changed as appropriate depending on how the step for bringing the test compound into contact with Vimentin or a functional fragment thereof has been performed; for example, when using a column packed with a solid phase carrier (e.g., bead resin) immobilized with the test compound, Vimentin molecules bind onto the solid phase carrier with the subsequent addition of a solution (sample) comprising Vimentin or a functional fragment thereof, provided that there is specific affinity between the two (do not bind in the absence of specific affinity). It is also possible to dissociate the bound Vimentin or a functional fragment thereof from the solid phase by a treatment such as altering the polarity of the buffer solution or further adding the test compound in excess, and then identify, or to extract with a surfactant and the like while remaining in a state bound onto the test compound on the solid phase, and then identify. As the method of identification, specifically, known techniques such as electrophoresis, immunoblotting and immunoprecipitation, which employ immunological reactions, chromatography, mass spectrometry, amino acid sequencing, and NMR, or combinations of these methods can be used. By determining whether or not Vimentin or a functional fragment thereof is captured onto the solid phase or contained in the column through fraction, or the extent thereof and the like, a judgment is made as to whether or not the test compound is capable of binding specifically to Vimentin, and a binding compound is selected.

Also, this step may be automated. For example, it is also possible to directly read data on various molecules obtained by two-dimensional electrophoresis, and identify the molecules on the basis of existing databases.

Furthermore, when Vimentin or a functional fragment thereof is used in the state of being expressed in cells, it is also possible to determine the presence or absence of binding of Vimentin or a functional fragment thereof and the test compound, and the degree of binding, by making use of various labeling techniques such as RI labeling and fluorescence labeling. “Contact of Vimentin or a functional fragment thereof and a test compound” in the screening method of the present invention also includes this mode. The contact conditions of cells and a test compound are set as appropriate depending on factors such as the cells to be used and the expression status of Vimentin or a functional fragment thereof in the cells. Also, whether or not Vimentin or a functional fragment thereof is expressed in the cells is preferably confirmed in advance using an antibody thereto and the like.

The effect of a compound obtained by the above described procedures as an anti-allergic agent can be confirmed by, for example, the suppressive effect on the secretion action of inflammation mediator and the like as found in a reference: Cell Calcium 26 (6), 261-269 (1999).

### EXAMPLES

The present invention is hereinafter described in more detail by means of the following Examples, which, however, are not to be construed as limiting the scope of the invention. Each compound, reagent and the like to be used are, unless otherwise specified, commercially available or can be prepared based on the known reports and the like.

### Example 1

### Synthesis of Intal-Immobilized Resin

### (1) Synthesis of 1,3-bis(2-carboxychromon-5-yloxy)-2-hydroxypropane

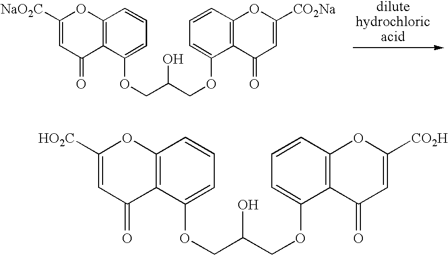

The disodium salt of 1,3-bis(2-carboxychromon-5-yloxy)-2-hydroxypropane (1 g) was dissolved in 50 ml of water. Dilute hydrochloric acid was added to obtain a pH of 3. The precipitated white crystal was collected by filtration and washed with water, ethanol, and ether. The crystal was dried under reduced pressure to yield a white crystal of 1,3-bis(2-carboxychromon-5-yloxy)-2-hydroxypropane (600 mg, yield 66%).

¹H-NMR (DMSO-d₆) δ: 4.30 (4H, d), 4.36 (1H, t), 5.32 (1H, bs), 6.86 (2H, s), 7.11 (2H, d), 7.17 (2H, d), 7.71 (2H, t).

### (2) Synthesis of 1,3-bis(2-carboxychromon-5-yloxy)-2-hydroxypropane-immobilized resin

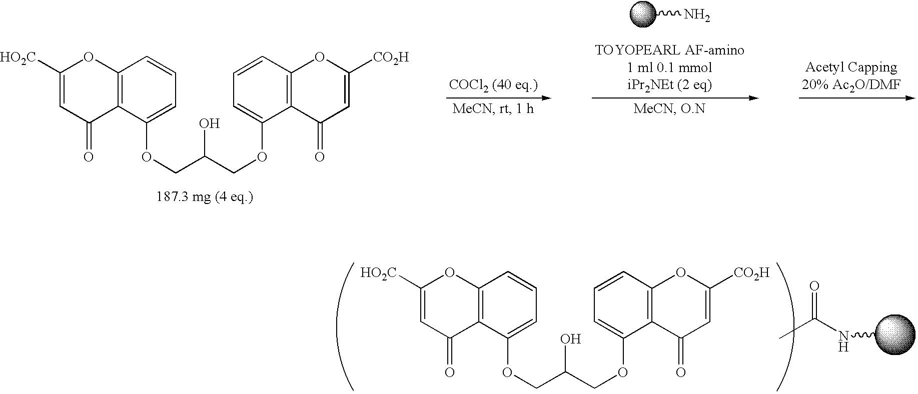

### (a) Immobilization of 1,3-bis(2-carboxychromon-5-yloxy)-2-hydroxypropane

To a suspension of 1,3-bis(2-carboxychromon-5-yloxy)-2-hydroxypropane (187.3 mg, 0.4 mmol) in acetonitrile (4.8 ml), a toluene solution of phosgene (1.65 mol/l, 24 μl) was added. The obtained reaction mixture as is was stirred for 1 hr at room temperature. After concentrating for 5 min. under reduced pressure on SpeedVac apparatus (approx. −90 kPa), the obtained concentrated solution was added to TOYO-Pearl resin (TSKgel AF-amino, 1 ml) and an suspension of diisopropylethylamine (35 μl, 0.2 mmol) in acetonitrile (5 ml), and the mixture was stirred overnight at room temperature. The obtained resin was washed with acetonitrile, saturated aqueous sodium hydrogencarbonate solution and water in this order, respectively 5 times.

### (b) Acetyl Capping

To the resin obtained from step (a) were added acetic anhydride (2 ml) and DMF (8 ml), the mixture was treated 1 hr at room temperature and then washed with DMF, 20% aqueous ethanol solution 5 times. The end point of the reaction was confirmed by ninhydrin reaction when the residual amino groups could not be observed any more. Finally, TOYO-Pearl resin was washed 5 times.

### Example 2

### Binding Experiments

### (1) Preparation of RBL-2H3 Cell Lysate

RBL-2H3 cells (250 mg) were mixed with mixture A (2.5 ml, 50 mM Sucrose, 300 μM sodium salt of N,N-diethyldithiocarbamate, 25 mM Urea, 2 mM dithiothreitol, 1 μM CaCl₂, 25 mM Tris-HCl, pH7.5), and the cells were disrupted by ultrasonication. The mixture was centrifuged at 9,000 rpm for 10 min., and the obtained supernatant was used as lysate. Note that all experiments were performed at 4° C. or on ice.

### (2) Binding Experiments

Binding experiments were performed using the immobilized resins with the test compound immobilized thereto, prepared in Production Example 1, and the RBL-2H3 cell lysate prepared in Example 2(1), according to procedures shown below.

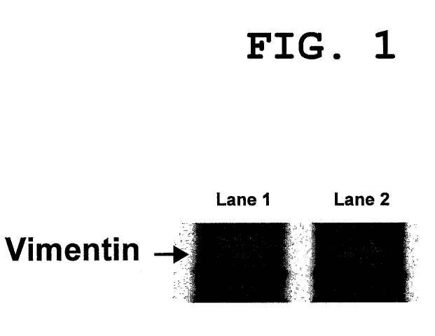

Respective resin (10 μl) and lysate (1 ml) were gently shaken at 4° C. for about 1 hour. Thereafter, centrifugal operation was performed, and respective supernatant was collected carefully. Then, respective supernatant was again mixed with a fresh compound-bound resin (10 μl). At this time, the separated compound-bound resin is stationarily stored at 4° C. as the resin for the first binding experiment. After the mixture was gently stirred for about 1 hour, centrifugal operation was performed, and the supernatant was removed. Subsequently, the compound-bound resin obtained in the second binding experiment and the resin obtained in the first binding experiment were carefully washed with mixture A about five times to remove substances other than the protein bound onto the resin as far as possible. To each compound-bound resin thus obtained, 30 μl of a loading buffer for SDS (nakalai Cat. No=30566-22, sample buffer solution for electrophoresis with 2-ME (2-mercaptoethanol) (2×) for SDS PAGE) was added, the mixture was stirred at 25° C. for 10 minutes. The sample solution thus obtained was separated using a commercially available SDS gel (BioRad readyGel J, 10% SDS, cat. NO=161-J371V), and the SDS gel was analyzed (FIG. 1). An electrophoregram of the sample solution comprising the protein which binds to the bound resin obtained in the first binding experiment (FIG. 1, lane 1), and an electrophoregram of the sample solution comprising the protein which binds to the bound resin obtained in the second binding experiment (FIG. 1, lane 2) were compared.

As a result, it was shown that Vimentin bound to the Intal-immobilized resin, and that the binding was a specific one because the binding was remarkably ascertained in the first binding experiment with compound-bound resin but observed little in the second binding experiment.

### Example 3

### Measurement of Kd Value Between Vimentin and Intal with Biacore

### <Synthesis of Intal PEG Amine>

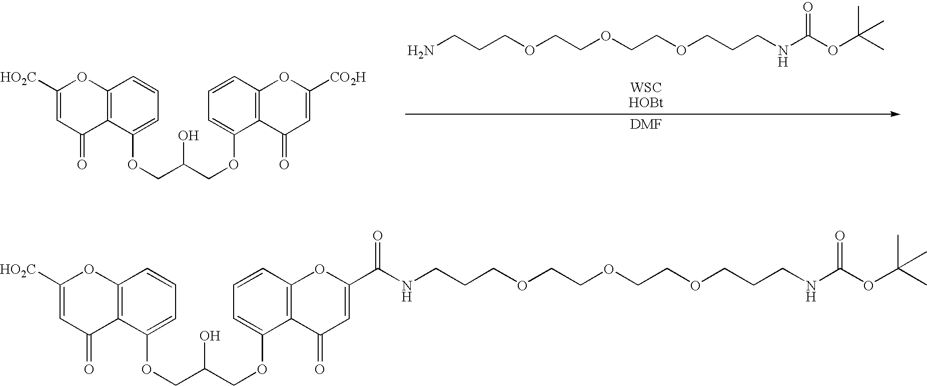

To a solution of 1,3-bis(2-carboxycromon-5-yloxy)-2-hydroxypropane (117 mg, 0.25 mmol) in dimethylformamide (2 ml) were added PEG amine (80 mg, 0.25 mmol), water-soluble carbodiimide (WSCD; 44 μl, 0.25 mmol) and HOBt (34 mg, 0.25 mmol), and the mixture was stirred overnight at room temperature. The crude reaction product was extracted, and then purified by thin layer chromatography. The PEGylated form was yielded as white solid (130 mg, 0.17 mmol, yield 67%).

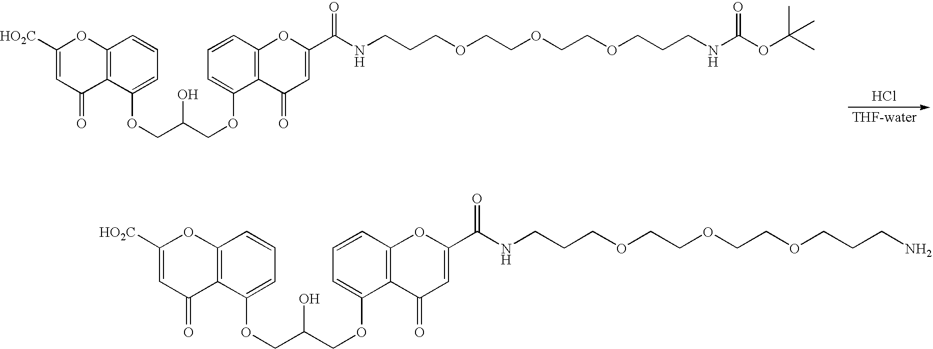

To a solution of PEGylated form (130 mg, 0.17 mmol) in water-tetrahydrofuran (1.5 ml; 1:2) was added concentrated hydrochloric acid (0.5 ml), and the mixture was stirred for 2 hr at room temperature. After concentration under reduced pressure, the crude reaction product was separated and purified by HP20. The PEGylated form was yielded as white solid (67 mg, 0.1 mmol, yield 59%).

¹H-NMR (CD₃OD) δ: 1.77-1.84 (4H, m), 3.01 (2H, t), 3.40 (2H, t), 3.46-3.53 (12H, m), 4.30-4.32 (5H, m), 6.90 (1H, d), 6.92 (1H, d), 7.04 (1H, d), 7.05 (1H, d), 7.49-7.66 (2H, m).

LC-MS: 671.1, purity 99% (254 nm)

<Synthesis of Intal-Immobilized Chip>

Intal PEG amine synthesized as above was immobilized to Biacore CM5 chip (#BR-1000-14), according to a method described in BIAapplications Handbook (published by Biacore).

Control data was obtained by using a chip to which 2-ethanolamine was immobilized by the same way.

### <Preparation of Vimentin Sample>

As Vimentin, Standard Human Vimentin (cat. No. 62015) purchased from PROGEN Biotechnik was subjected to buffer exchange by Running buffer(0.25 M Sucrose, 25 mM Tris-HCl pH7.5, 0.3 mM sodium N,N-diethyldithiocarbamate, 25 mM urea, 1 mM calcium chloride) and used to measure Kd.

### <Measurement of Kd Value>

The measurement of Kd was performed by SPR (surface plasmon resonance) spectrum measurement on Biacore3000 (Biacore). The above-mentioned Running buffer was run through the Intal-immobilized CM5 chip prepared by the above-mentioned procedure at 20 μl per min. Thereto, each sample which was prepared by gradually diluting the Vimentin prepared by the above-mentioned procedure (0.29 nM to 4.6 μM) was infused for 5 min., and the obtained SPR spectrum was analyzed by BIAevaluation software Ver. 4.1 (Biacore) to get 56 nM of Kd.

From above results, it was demonstrated that the interaction between Intal and Vimentin was specific binding.

### INDUSTRIAL APPLICABILITY

According to a method of screening of the present invention, one can efficiently screen for a compound having a mechanism of action and pharmacological activity similar to Intal. A compound obtainable by a method of screening of the present invention can be useful as a pharmaceutical agent which has an effect over Intal or is alternative thereto.

This application is based on patent application No. 318833/2005 filed in Japan, the contents of which are hereby incorporated in full by reference.

1. A method of screening for a compound useful for the treatment of an allergic disease, which comprises a step of determining whether a test compound specifically binds to Vimentin or a functional fragment thereof.

2. A method of screening for a compound useful for the treatment of an allergic disease, which comprises the following steps:
(1) contacting Vimentin or a functional fragment thereof with test compounds,
(2) determining whether the test compounds specifically bind to Vimentin or a functional fragment thereof, and
(3) selecting a test compound which specifically binds to Vimentin or a functional fragment thereof in the step (2) above.

3. A method of screening for a compound useful for the treatment of an allergic disease, which comprises the following steps:
(1) contacting a protein having an amino acid sequence of SEQ ID NO:2 or a fragment thereof with test compounds,
(2) determining whether the test compounds specifically bind to the protein or a functional fragment thereof, and
(3) selecting a test compound which specifically binds to the protein or a functional fragment thereof in the step (2) above.

4. A method of screening for a compound useful for the treatment of an allergic disease, which comprises the following steps:
(1) contacting, with test compounds, a protein having an amino acid sequence with deletion, substitution or addition of one or more amino acids in the amino acid sequence of SEQ ID NO:2 and binding to the following compound 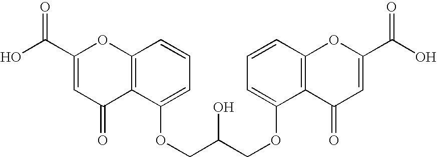
or a fragment thereof,
(2) determining whether the test compounds specifically bind to the protein or a functional fragment thereof, and
(3) selecting a test compound which specifically binds to the protein or a functional fragment thereof in the step (2) above.

5. A compound useful for the treatment of an allergic disease, obtainable by the method of screening according to claim 1.

6. A pharmaceutical composition comprising a compound which specifically binds to Vimentin as an active ingredient.

7. A pharmaceutical composition comprising a compound which controls the expression of Vimentin as an active ingredient.

8. A pharmaceutical composition comprising a compound which controls the activity of Vimentin as an active ingredient.

9. The pharmaceutical composition according to claim 6, which is used for the treatment of an allergic disease.

10. A compound represented by the formula (I): 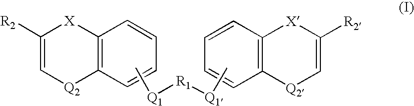
[wherein,
R₁ is optionally substituted divalent hydrocarbon;
R₂ and R_{2′} are the same or different and each is carboxylic acid, amide or ester (which is optionally substituted by lower alkyl) or tetrazole;
Q₁ and Q_{1′} are the same or different and each is O, S, NH or CH₂;
Q₂ and Q_{2′} are the same or different and each is C═O, C═S, C═NH, C═NOH, C═NOR₃, O, alkylene or NH;
X is CH₂, O, S, NH or NR₃ (wherein, R₃ is optionally substituted alkyl or cycloalkyl)],
or a pharmaceutically acceptable salt thereof,
provided that the following compound is excluded:

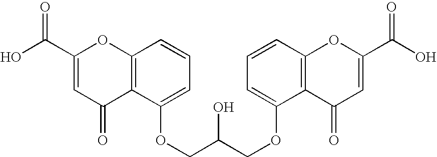

11. A therapeutic agent for an allergic disease, which comprises the compound according to claim 10 or a pharmaceutically acceptable salt thereof.

12. A compound useful for the treatment of an allergic disease, obtainable by the method of screening according to claim 2.

13. A compound useful for the treatment of an allergic disease, obtainable by the method of screening according to claim 3.

14. A compound useful for the treatment of an allergic disease, obtainable by the method of screening according to claim 4.

15. The pharmaceutical composition according to claim 7, which is used for the treatment of an allergic disease.

16. The pharmaceutical composition according to claim 8, which is used for the treatment of an allergic disease.
